(19) United States
(12) Patent Application Publication
(10) Pub. No.: US 2007/0276151 A1
(43) Pub. Date: Nov. 29, 2007

(54) Method For Preparing Asymmetric Linear Carbonate

(76) Inventors: Min-Hyuk Lee, Kyonggi-Do (KR); Jeong-Ho Park, Kyonggi-Do (KR); Seung-Hwan Kim, Kyonggi-Do (KR); Seong-Sam Park, Kyonggi-Do (KR)

Correspondence Address: BIRCH STEWART KOLASCH & BIRCH, PO BOX 747, FALLS CHURCH, VA, 22040-0747, US

(21) Appl. No.: 11/667,413

(22) PCT Filed: Oct. 19, 2005
(86) PCT No.: PCT/KR05/03488

§ 371 Date: May 9, 2007

(30) Foreign Application Priority Data
Nov. 16, 2004 (KR) 10-2004-0093604

(51) Int. Cl. C07C 69/96

(52) U.S. Cl. 558/260

(57) ABSTRACT

A method for preparing asymmetric linear carbonate useful as an electrolyte for lithium secondary battery is disclosed. The method comprises the steps of: removing methyl acetate by a distillation while carrying out a transesterification of dimethyl carbonate with acetate compound in the presence of a basic catalyst; and separating the asymmetric linear carbonate from the transesterification product.

### TECHNICAL FIELD

This invention relates to a method for preparing asymmetric linear carbonate, and more specifically to a method for preparing asymmetric linear carbonate useful as a solvent for lithium secondary battery, etc.

### BACKGROUND ART

Asymmetric linear carbonate such as ethyl methyl carbonate (EMC) has been generally used as a solvent (electrolyte) for lithium secondary battery, and the lithium secondary battery using the asymmetric linear carbonate as an electrolyte has improved characteristics including increased energy density, increased discharge capacity, longer life cycle and higher safety performance in comparison with the battery using a conventional electrolyte. Accordingly, the asymmetric linear carbonate is mainly used as an electrolyte for lithium secondary battery. A conventional method of preparing the asymmetric linear carbonate is an esterification of alkyl chloroformate with alcohol in the presence of a basic catalyst, but the method has problems in that the esterification reaction is very reactive and requires highly toxic starting materials such as phosgene and bisphenol A. As a method to complement these problems, a method for preparing asymmetric linear carbonate is disclosed in Japanese Laid-Open patent Publication No. H6-166660. The method uses a transesterification of symmetric linear carbonate with alkyl alcohol in the presence of a basic catalyst such as metal carbonate salt. However, the method has problems in that the catalyst activity and the reaction yield are low, and the method requires separation and purification process of the final target compound, for example, ethyl methyl carbonate from the reaction product including three linear carbonate compounds and two alcohol compounds. A method for preparing the asymmetric linear carbonate disclosed in U.S. Pat. No. 5,962,720 uses a transesterification of two different symmetric carbonates in the presence of a basic catalyst such as a Group 1A or Group 2A metal alkoxide salt or a Group 1A or Group 2A metal amide salt which is a nucleophilic or reductive catalyst. The method has advantages in that the reaction yield is high, and alcohol is not necessary for the transesterification, but the method has disadvantages in that the basic catalyst should be separated from the reaction product with an Alumina or Silica Gel column, and the trace of water or alcohol in the reactants should be eliminated out before the transesterification reaction to prevent the deterioration of a catalyst activity due to water or alcohol in the reactants. A method disclosed in Japanese Laid-Out Patent publication Nos. 2000-344715 and 2000-344718 produces the asymmetric linear carbonate in the presence of water or alcohol, by using the oxides of rare earth metals of a Group 3B. However, the method has the problems in that the reaction is carried out at high pressure of 5 to 10 atm, and for long time interval of 200 hours or more.

### DISCLOSURE OF INVENTION

### Technical Problem

Therefore, it is an object of the present invention to provide a method for preparing asymmetric linear carbonate, in which a catalyst activity is not deteriorated in spite of the existence of water or alcohol, and accordingly the asymmetric linear carbonate can be produced with high yield and high purity in a short time.

It is other object of the present invention to provide a method for preparing asymmetric linear carbonate, in which the reactants and reaction process are easily controlled, and the asymmetric linear carbonate can be mass-produced on a large scale.

### Technical Solution

To accomplish these objects, the present invention provides the method for preparing asymmetric linear carbonate, which comprises the steps of: removing methyl acetate by a distillation while carrying out a transesterification of dimethyl carbonate with acetate compound in the presence of a basic catalyst; and separating the asymmetric linear carbonate from the transesterification product. Wherein, the preferable basic catalyst includes lithium methoxide, lithium ethoxide, sodium methoxide, lithium amide, calcium hydride and the mixtures thereof.

### MODE FOR THE INVENTION

A more complete appreciation of the invention, and many of the attendant advantages thereof, will be better appreciated by reference to the following detailed description.

In order to prepare the asymmetric linear carbonate according to the present invention, a transesterification of dimethyl carbonate with acetate compound is carried out according to the following Reaction 1 in the presence of a basic catalyst.

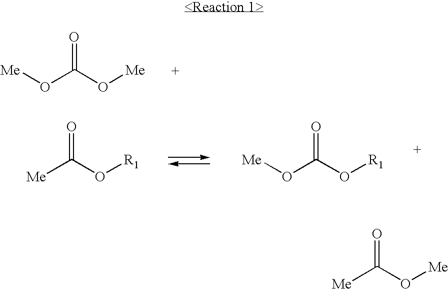

In Reaction 1, R₁ is a linear alkyl group, a branched alkyl group, or a cyclic alkyl group, and preferably a C2-C10 linear alkyl group, a C3-C10 branched alkyl group, or a C5-C10 cyclic alkyl group. The preferable acetate compound includes ethyl acetate, propyl acetate, isopropyl acetate, n-butyl acetate, t-butyl acetate, the mixtures thereof, and so on, having a C2-C4 linear alkyl group. (“C2-C10” represents the number of carbon atoms is 2 to 10.)

Preferably, dimethyl carbonate and acetate compound (dimethyl carbonate:acetate compound) are used in the molar ratio of 1:10 to 10:1, and more preferably in the molar ratio of 1:1 to 1:2, and most preferably in the molar ratio of 1:1 to 1:1.5 to maximize the reaction yield. If the amounts of dimethyl carbonate and acetate compound are beyond the above mentioned range, the reaction yield of the final product, namely asymmetric linear carbonate decreases.

The basic catalyst for the transesterification reaction may include nucleophilic or reductive metal salt. The preferable basic catalyst includes alkoxide salt of a group 1A or a group 2A metal, amide salt of a group 1A or a group 2A metal, metal hydride, more preferably hydride of a group 1A or a group 2A metal, and the mixtures thereof. Examples of the basic catalyst include lithium methoxide(LiOCH₃), lithium ethoxide(LiOC₂H₅), sodium methoxide(NaOCH₃), lithium amide(LiNH₂), calcium hydride(CaH₂) and so on. The preferable amount of the catalyst in the present invention is 0.01 to 10 weight % with respect to the total amount of dimethyl carbonate and the acetate compound, and more preferably 0.1 to 5 weight %. If the amount of the catalyst is less than 0.01 weight % with respect to the total amount of dimethyl carbonate and the acetate compound, the reaction rate decreases. If the amount of the catalyst is more than 10 weight % with respect to the total amount of dimethyl carbonate and the acetate compound, it is economically unfavorable without additional advantages.

The byproduct of the transesterification reaction, namely, methyl acetate, is removed by a distillation, preferably by a fractional distillation, during the transesterification reaction. In the transesterification reaction of the present invention, the acetate compound is converted into methyl acetate, and the reaction product includes three linear carbonate compounds and two acetate compounds. The byproduct, methyl acetate, is removed by condensing the vapor which contains a lot of methyl acetate at the top plate of a fractional distillation apparatus. The fractional distillation and the transesterification reaction are carried out at the same time in a conventional batch reactor, which is equipped with a fractional distillation apparatus preferably having the number of theoretical plates of 30, and more preferably having the number of theoretical plates of 50. The temperature of the top plate of the fractional distillation apparatus can be maintained at the temperature of more than 58° C., which is a boiling point of methyl acetate. If the liquid, which is a condensation product of the vapor and is condensed at the top plate of the fractional distillation apparatus, is partially refluxed, a condensed liquid including methyl acetate of higher purity can be obtained. Methyl acetate obtained by such process can be reused. The boiling point of methyl acetate (58° C.) is lower by more than 30° C. than the boiling point of dimethyl carbonate (90° C.), and methyl acetate forms an azeotrope with water and methanol. Therefore, water and alcohol are also easily removed with methyl acetate from the reaction product.

The transesterification reaction temperature is preferably 50° C. to 250° C., and more preferably 70° C. to 120° C. If the reaction temperature is less than 50° C., the productivity of the reaction decreases because of the slowdown of reaction rate. If the reaction temperature is more than 250° C., the reactants may be decomposed, and various byproducts can be produced. The pressure of the transesterification reaction can be widely varied without limitation, but the transesterification reaction can be preferably carried out in atmospheric pressure. The reaction time of the transesterification reaction can also be widely varied without limitation. Preferably, the transesterification reaction can be carried out for 0.1 hour to 10 hour, and more preferably for 0.5 hour to 4 hour. The transesterification reaction can be carried out until the composition of reaction product is not changed. The variation in the composition of reaction product can be determined by sampling the reaction product periodically during the reaction, and by analyzing the sampled reaction product with a gas chromatography.

After the transesterification reaction, the asymmetric linear carbonate is separated from the transesterification product. After completion of the transesterification, the reaction product, from which methyl acetate is removed, includes preferably only three linear carbonate. The separation of the asymmetric linear carbonate from the reaction product can be carried out by using a conventional distillation process at atmospheric or reduced pressure. When the reaction product is distillated at atmospheric or reduced pressure, compounds in the reaction product are successively distilled according to their boiling points. For example, after the transesterification of dimethyl carbonate with ethyl acetate, the reaction product is successively distilled in order of dimethyl carbonate (boiling point: 90° C.), ethyl methyl carbonate, and diethyl carbonate (boiling point: 127° C.). Accordingly, ethyl methyl carbonate having the purity of more than 99.9% can be obtained, and the separated dimethyl carbonate and diethyl carbonate can be recovered and reused.

Hereinafter, the preferable examples are provided for better understanding of the present invention. However, the present invention is not limited to the following examples.

### EXAMPLE 1

1.1 mole (99 g) of dimethyl carbonate (DMC), 1.3 mole (114.4 g) of ethyl acetate (EA), 0.21 g (0.1 weight %) of LiOCH₃ as a catalyst were added to a 500 ml reaction flask equipped with a fractional distillation apparatus having the number of theoretical plates of 50 and containing a magnetic stirring bar. The mixtures were stirred while being heated to 77° C. for carrying out the transesterification reaction. During the initial stage of the reaction, all of the liquid condensed at the top plate of the fractional distillation apparatus was refluxed. After 30 minutes of reaction, the temperature of the top plate reached to 58° C., which is the boiling point of methyl acetate, and the condensed liquid was refluxed with the reflux ratio of 3, and the unrefluxed condensed water was removed. After 3 hours of reaction, it was confirmed by a gas chromatography that the acetate compound did not exist in the reaction product, and then the reaction was completed. After completion of the reaction, the reaction product was analyzed with a gas chromatography. The gas chromatography analysis indicated that the molar ratio of dimethyl carbonate:ethyl methyl carbonate:diethyl carbonate was determined to be 1:2:1, and the reaction yield of ethyl methyl carbonate was 50% with respect to dimethyl carbonate (DMC). Next, the temperature of the reaction product was elevated to 110° C., and the reaction product was fractional distillated with the reflux ratio of equal or more than 5 to obtain ethyl methyl carbonate having the purity of 99.9% (Distillation yield: 85%). The moisture content of the obtained ethyl methyl carbonate was 50 ppm, which was measured by Karl Fisher titration.

### EXAMPLE 2

Except for using 0.5 weight % (1.07 g) of LiNH₂ instead of 0.1 weight % of LiOCH₃, and carrying out the reaction for 4 hours, the asymmetric linear carbonate was prepared in the same manner as described in Example 1. After completion of the reaction, the reaction product was analyzed by a gas chromatography. The gas chromatography analysis indicated that the molar ratio of dimethyl carbonate:ethyl methyl carbonate:diethyl carbonate in the reaction product was determined to be 1:1.8:1, and the reaction yield of ethyl methyl carbonate was 47% with respect to dimethyl carbonate. Next, the reaction product was fractional distillated with the reflux ratio of equal or more than 5 to obtain ethyl methyl carbonate having the purity of 99.9% (Distillation yield: 78%). The moisture content of the obtained ethyl methyl carbonate was 50 ppm, which was measured by Karl Fisher titration.

As described above, the method for preparing the asymmetric linear carbonate according to the present invention can produce the asymmetric linear carbonate of high purity. In the present invention, the catalyst activity is not deteriorated in spite of the existence of water or alcohol in the reactants, which results in the production of the asymmetric linear carbonate with high yield in a short time. In addition, the reactants and reaction process can be easily controlled, and the asymmetric linear carbonate can be mass-produced on a large scale.

While this invention has been shown and described with reference to certain preferred embodiments thereof, it will be understood by those skilled in the art that various changes in form and details may be made therein without departing from the spirit and scope of the invention as defined by the appended claims.

1. A method for preparing asymmetric linear carbonate comprising the steps of:
removing methyl acetate by a distillation while carrying out a transesterification of dimethyl carbonate with acetate compound in the presence of a basic catalyst; and
separating the asymmetric linear carbonate from the transesterification product.

2. The method for preparing asymmetric linear carbonate of claim 1, wherein the acetate compound is selected from the group consisting of ethyl acetate, propyl acetate, isopropyl acetate, n-butyl acetate, and t-butyl acetate.

3. The method for preparing asymmetric linear carbonate of claim 1, wherein the basic catalyst is nucleophilic or reductive metal salt.

4. The method for preparing asymmetric linear carbonate of claim 1, wherein the basic catalyst is selected from the group consisting of lithium methoxide, lithium ethoxide, sodium methoxide, lithium amide, calcium hydride and the mixtures thereof.

5. The method for preparing asymmetric linear carbonate of claim 1, wherein the molar ratio of dimethyl carbonate and the acetate compound is 1:1 to 1:2.

6. The method for preparing asymmetric linear carbonate of claim 1, wherein the step of separating the asymmetric linear carbonate is carried out by a distillation of the transesterification product.
